(19) United States
(12) Patent Application Publication
(10) Pub. No.: US 2025/0001022 A1
(43) Pub. Date: Jan. 2, 2025

(54) STATIC APPARATUS AND METHOD FOR DELIVERING NANOSECOND PULSED ELECTRIC FIELDS TO CORONAVIRUS FOR SELECTIVE CORONAVIRUS STERILIZATION

(71) Applicant: Government of the United States as represented by the Secretary of the Air Force, Wright-Patterson AFB, OH (US)

(72) Inventors: Ronald Barnes, Indian Harbour Beach, FL (US); Jody Cantu, Abingdon, MD (US); Ibtissam Echchgadda, San Antonio, TX (US); Bryan Gamboa, San Antonio, TX (US); Bennett Ibey, San Antonio, TX (US)

(21) Appl. No.: 18/757,931

(22) Filed: Jun. 28, 2024

Related U.S. Application Data
(60) Provisional application No. 63/511,637, filed on Jun. 30, 2023.

(51) Int. Cl. A61L 2/03

(52) U.S. Cl. CPC A61L 2/035

(57) ABSTRACT

An apparatus and method for deactivating coronavirus from a liquid having entrained coronavirus. The apparatus comprises a nanosecond pulsed electric field (nsPEF) generator which imparts nsPEF energy to the through the solution to the coronavirus. The nsPEF disrupts the viral membrane, deactivating the virus. The apparatus may be static using a reservoir for batch treatment of viral solution, or may be dynamic using a closed loop for continuous treatment of viral solution. Total energy input may be less than 3500 KVns providing an efficient and effective process. The method and apparatus according to the present invention advantageously avoid boiling.

### CROSS REFERENCE TO RELATED APPLICATION

This application claims priority to and the benefit of U.S. application 63/511,637 filed Jun. 30, 2023, the disclosure of which is incorporated herein by reference.

### RIGHTS OF THE GOVERNMENT

The invention described herein may be manufactured, licensed and used by and for the Government of the United States for all governmental purposes without the payment of any royalty.

### FIELD OF THE INVENTION

The present invention relates to an apparatus and method useful to efficaciously deactivate coronavirus using electrical energy input and more particularly to such an apparatus and method which uses nanosecond pulsed electric fields (nsPEF) to efficaciously deactivate coronavirus.

### BACKGROUND OF THE INVENTION

The response to the COVID-19 pandemic revealed a major need for physical sterilization techniques, which should consist of technologies that are non-harmful to humans and, therefore, can be deployed in occupied public spaces. One technology to fill this void uses electromagnetic fields to neutralize microbes within Institute of Electrical and Electronics Engineers (IEEE) safety standards. Recent studies suggest that electromagnetic waves can elicit a resonant effect on viruses, causing inactivation. Specifically, the exposure of microbes of sizes <1 μm in diameter, which includes coronaviruses, to a pulse with a high E-field strength can induce complex mechanical and physical breakdown of the material, including complete destruction of the virus. Exposure to high-power, short duration electric pulses, such as nsEPs, causes the permeabilization of cellular membranes. Specifically, nsEP cellular studies showed that exposures can injure cell membranes by inducing permeabilization, which can result in the free passage of molecules across the plasma membrane.

Coronaviruses are composed of a single-stranded ribonucleic acid (RNA) core surrounded by a lipid envelope with embedded viral glycoproteins (nucleocapsid, spike, envelope, haemagglutinin-esterase, and integral membrane proteins). Dissolution of the viral envelope renders it neutralized. Accordingly, a sterilization technique may target the viral envelope to induce breakdown. The lipid envelope is partially derived from portions of the host cell membrane (phospholipids) and, therefore, could be susceptible to the same nsEP-derived forces that permeabilize all biological lipid membranes. Suitable effects are described by the present inventors in Cantu, J. C., Barnes, R. A., Gamboa, B. M. et al. Effect of nanosecond pulsed electric fields (nsPEFs) on coronavirus survival. AMB Expr 13, 95 (2023). https://doi.org/10.1186/s13568-023-01601-3, published 9 Sep. 2023, the disclosure of which is incorporated herein by reference.

Various attempts have been made to address the need to conveniently sterilize and deactivate coronavirus. For example, US 2022/0088403 to Voloshin-Sela et al. discloses a method directed to inhibiting a coronavirus by exposing a coronavirus cell to an alternating electric field for 24 or 48 hours. U.S. Pat. No. 11,202,673 to Jacobowitz et al. discloses a electrodynamic radiative electroporation requiring plural antennae and whole body treatment. These attempts in the art require extended duration treatments and/or complex systems which may be difficult to manage and properly operate. Furthermore, these attempts may produce thermal loads which are undesirable in both laboratory and clinical settings. Alternatively, ultraviolet and chemical treatments have proven effective but can be unsafe in the presence of humans.

The present invention overcomes the problem of coronaviral transmission and the problems arising from the attempts in the prior art. The present invention provides a flexible and compact apparatus and method useful to efficaciously deactivate coronavirus.

### SUMMARY OF THE INVENTION

In one embodiment the invention comprises a static method of viral sterilization comprising the steps of: providing a coronavirus in a reservoir; providing two spaced apart electrodes in electrical communication with the coronavirus and being connected to an electrical power supply; and applying an effective amount of nanosecond duration electric pulses (nsEPs) from the power supply to thereby substantially deactivate the coronavirus.

In one embodiment the invention comprises a method of static selective coronavirus sterilization comprising the steps of: providing 400 microliters to 500 microliters of bovine coronavirus in a reservoir; providing two spaced apart electrodes in electrical communication with the coronavirus and being connected to an electrical power supply; and applying an effective amount of nanosecond duration monopolar electric pulses (nsEPs) from the power supply to thereby substantially deactivate the coronavirus.

In one embodiment the invention comprises an apparatus for viral sterilization of coronavirus comprising: a reservoir for containing a coronavirus solution therein; and a power supply in electrical communication with two spaced apart electrodes configured to apply an electric current to a coronavirus solution when contained within the reservoir, the power supply being able to provide upon demand a nanosecond duration electric pulses (nsEPs) effective to sterilize an object disposed in the reservoir from a coronavirus.

In one embodiment the invention comprises an apparatus for dynamically sterilizing a viral solution without boiling, the apparatus comprising: a reservoir for containing a coronavirus therein; at least one stage for deactivating the coronavirus, each at least one stage having a power supply having a voltage amplitude of 1 kV/cm to 25 kV/cm and a nanosecond pulsed electric field (nsPEF) generator configured to impart a 1 ns to 1000 nsPEF to the solution; a power supply for supplying electric power to the nsPEF; a pipe for communicating the viral solution from the reservoir through the stage and back into the reservoir thereby forming a closed loop; and a pump for urging flow of the solution through the closed loop.

In one embodiment the invention comprises a method of dynamically sterilizing a liquid viral solution without boiling, the method comprising the steps of: providing an apparatus comprising a reservoir for containing a coronavirus therein, at least one stage for deactivating the coronavirus, each at least one stage having a power supply having a voltage amplitude of 1 kV/cm to 25 kV/cm and a nanosecond pulsed electric field (nsPEF) generator configured to impart a 1 ns to 1000 nsPEF across two spaced apart paired electrodes to the solution, a power supply for supplying electric power to the nsPEF, a pipe for communicating the viral solution from the reservoir through the stage and back into the reservoir thereby forming a closed loop and a pump for urging flow of the solution through the closed loop; energizing the pump to operably cause a fluid flow of the liquid viral solution from the reservoir, through the at least one stage and back into the reservoir through the closed loop; and energizing the nsPEF generator to impart an effective amount of nsPEF energy to the solution to thereby deactivate coronavirus in proximity to the paired electrodes.

In one embodiment the invention comprises a method of sanitizing blood from coronavirus, the method comprising the steps of: providing an human comprising blood containing a coronavirus therein, at least one stage for deactivating the coronavirus, each at least one stage having a power supply having a voltage amplitude of 1 kV/cm to 25 kV/cm and a nanosecond pulsed electric field (nsPEF) generator configured to impart a 1 ns to 1000 nsPEF across two spaced apart paired electrodes to the solution, a power supply for supplying electric power to the nsPEF, a pipe for communicating the viral solution from the human through the stage and back into the human thereby forming a closed loop and a pump for urging flow of the solution through the closed loop; energizing the pump to operably cause a fluid flow of the liquid viral solution from the human, through the at least one stage and back into the human through the closed loop; and energizing the nsPEF generator to impart an effective amount of nsPEF energy to the solution to thereby deactivate coronavirus in proximity to the paired electrodes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation of one embodiment of a system according to the invention.

FIG. 2 is a schematic representation of one embodiment of a system according to the invention.

FIG. 2A is a schematic vertical sectional view of an alternative embodiment of a pipe usable with the system of FIG. 2.

FIG. 3A shows oscilloscope traces of the applied voltage used to deliver an E-field amplitude of 12.5 kV/cm MP (orange) and 25 kV/cm MP (blue) pulses.

FIG. 3B shows oscilloscope traces of the applied voltage oscilloscope traces of the applied voltage used to deliver an E-field amplitude of 12.5 kV/cm BP (green) or 25 kV/cm BP (red) pulses

FIG. 3C is a Superimposition of the MP and BP pulses in FIG. 3A and FIG. 3B.

FIG. 3D is a model of the predicted E-field distribution throughout an electroporation cuvette with the E-Field being uniform throughout the cuvette media.

FIG. 4A is a model of the predicted cuvette temperature increase for a 100 nsEP pulse duration at two different voltages.

FIG. 4B is a model of the predicted cuvette temperature increase for a 100 nsEP pulse duration at two different voltages.

FIG. 5A is a 10× photomicrograph showing cytopathic effects of control HRT-18G host cells.

FIG. 5B is a 10× photomicrograph showing cytopathic effects of virus infected HRT-18G host cells.

FIG. 5C is a 20× photomicrograph showing cytopathic effects of HRT-18G h control host cells.

FIG. 5D is a 20× photomicrograph showing cytopathic effects of virus infected HRT-18G host cells.

FIG. 6A is a representation of the temperature response to orders of magnitude of 12.5 kV/cm monopolar pulses to a host.

FIG. 6B is a representation of the temperature response to orders of magnitude of 25 kV/cm monopolar pulses to a host.

FIG. 6C is a representation of the temperature response to orders of magnitude of 12.5 kV/cm bipolar pulses to a host.

FIG. 6D is a representation of the temperature response to orders of magnitude of 25 kV/cm bipolar pulses to a host.

FIG. 6E is a chart of the data in FIGS. 6A-6D, showing the standard error of the mean.

FIG. 6F is a comparison of the measured and modeled temperature increases for 12.5 kV/cm and 25 kV/cm pulses at four different orders of magnitude pulse counts.

FIG. 7A is a performance chart of Log 10 infectivity for 12.5 kV/cm monopolar pulses at four different orders of magnitude pulse counts.

FIG. 7B is a performance chart of percentage inactivation for 12.5 kV/cm monopolar pulses at four different orders of magnitude pulse counts.

FIG. 7C is a performance chart of Log 10 infectivity for 12.5 kV/cm monopolar pulses at four different orders of magnitude pulse counts.

FIG. 7D is a performance chart of percentage inactivation for 12.5 kV/cm monopolar pulses at four different orders of magnitude pulse counts.

FIG. 8A is a performance chart of Log 10 infectivity for 12.5 kV/cm bipolar pulses at four different orders of magnitude pulse counts.

FIG. 8B is a performance chart of percentage inactivation for 12.5 kV/cm bipolar pulses at four different orders of magnitude pulse counts.

FIG. 8C is a performance chart of Log 10 infectivity for 12.5 kV/cm bipolar pulses at four different orders of magnitude pulse counts.

FIG. 8D is a performance chart of percentage inactivation for 12.5 kV/cm bipolar pulses at four different orders of magnitude pulse counts.

FIG. 9A is a graph of the inactivation percentage as a function of temperature.

FIG. 9B is a graph of the inactivation percentage as a function of pulse count.

FIG. 9C is chart of the data in FIGS. 9A-9B, showing the standard error of the mean.

FIG. 10A is a graph of applying constant voltage at constant pulse duration according to a process of the invention, showing a schematic linear temperature increase throughout the process.

FIG. 10B is a graph of applying both stepwise and monotonically decreasing voltage at constant pulse duration according to a process of the invention.

FIG. 10C is a graph of applying both stepwise and monotonically decreasing pulse duration at constant voltage according to a process of the invention.

FIG. 10D is a graph of applying both stepwise and monotonically decreasing pulse durations and stepwise and both monotonically decreasing voltage according to a process of the invention.

FIG. 11A is an analytical model of the decay of an applied monopolar nsPEF and bipolar nsPEF. The blue line and yellow line represent monopolar and bipolar modeling results, respectively.

FIG. 11B is an analytical model of viral membrane charge responsive to the applied monopolar nsPEF and bipolar nsPEF of FIG. 11A. The blue line and yellow line represent monopolar and bipolar modeling results, respectively.

FIG. 11C is an analytical model of the displacement of a viral cell due to an applied monopolar nsPEF and bipolar nsPEF. The blue line and red line represent monopolar and bipolar modeling results, respectively.

FIG. 11D is an analytical model of the velocity of a viral cell due to an applied monopolar nsPEF and bipolar nsPEF. The blue line and red line represent monopolar and bipolar modeling results, respectively.

FIG. 11E is an analytical model of the jerk of a viral cell due to an applied monopolar nsPEF and bipolar nsPEF. The blue line and red line represent monopolar and bipolar modeling results, respectively.

### DETAILED DESCRIPTION OF THE INVENTION

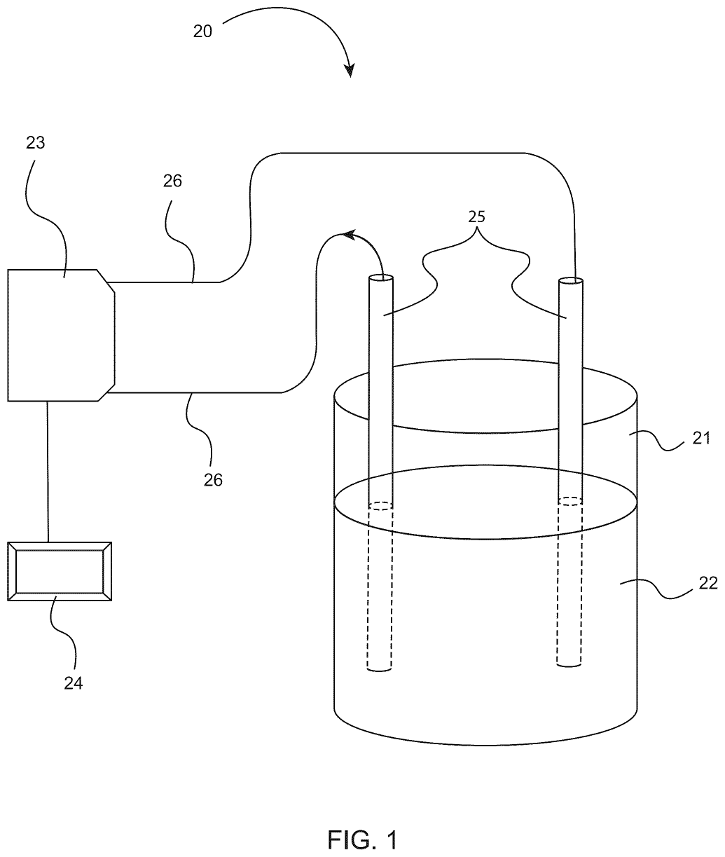

Referring to FIG. 1, in one embodiment the invention comprises an apparatus 20 for viral sterilization of coronavirus. A static apparatus 20 comprises a reservoir 21 for holding a solution 22 containing coronavirus therein, a pair of spaced apart electrodes 25 for delivering a nsPEF to the solution 22, a nsPEF generator 23 for generating the nsPEF charge and a power supply 24 for providing power to the nsPEF generator 23. One of skill will understand the solution 22 described below is a liquid solution 22 without phase change to vapor or the solution 22 may be a gas throughout the process described and claimed herein.

Examining the invention in more detail, the power supply 24 may be mains power or battery power as is known in the art. The power supply 24 may be integral with or otherwise in electrical communication with the nsPEF generator 23. The power supply 24 may be switched so that the nsPEF generator 2 can be on or off as desired.

The nsPEF generator 23 provides a nsPEF pulse, defined herein as an electric pulse having a duration of 1 ns to 1000 ns and an amplitude of 1 kV/cm to 25 kV/cm, where the spacing between electrodes 25 is measured in cm. The nsPEF generator 23 may be a Marx generator, using a Marx capacitor bank. A Marx generator has a bank of capacitors which are charged in parallel and then discharged in series. Each capacitor in the bank is isolated on both the high and low sides by a charging element, such as a resistor or inductor. The charging element serves the dual roles of defining the charge rate of the capacitor bank, and isolating each capacitor so that when switched into a series combination, the RC time constant of the parallel circuit is much greater than the RC time constant of the series discharge circuit. Therefore the voltage on each capacitor does not have time to fall off before the series circuit is fully formed and the pulse is delivered to the load, i.e. the solution 22. A single nsPEF generator 23 may be used to generate a monopolar (MP) nsPEFs, while two nsPEF generators 23 may be used to generate bipolar (BP) nsPEFs. Plural pulses are distinct and separated in time by at least 1 ns.

The nsPEF generator 23 may provide individual pulse periods ranging from 10 ns to 1000 ns. BP nsPEFs may be symmetric or asymmetric. By way of nonlimiting example, an BP nsPEF having a total period of 1000 ns may have periods of 500 ns+500 ns, 300 ns+700 ns, 800 ns+200 ns, 450 ns+550 ns, 550 ns+450 ns, etc. Similarly, the voltage amplitudes and pulse durations may be the same or different throughout the pulses.

One of skill will understand total energy imparted to the solution 22 will be varied as needed depending upon pulse count, pulse duration and pulse voltage amplitude. These parameters have the same correlations for both MP and BP pulses.

BP nsPEFs may have an individual pulse period ranging from 250 ns+250 ns to 350 ns+350 ns and preferably 300 ns+300 ns for a total of 600 ns duration. MP nsPEFs may have an individual pulse period ranging from 500 ns to 700 ns and preferably 600 ns for a total of 600 ns duration. The nsPEF generator 23 may provide MP or BP nsPEFs at a frequency of 0.9 Hz to 1.1 Hz and preferably 1 Hz.

As frequency and/or pulse period decrease, the nsPEF generator 23 may compensate by increasing the applied voltage. A MP or BP voltage may range from 1 kV/cm to 25 kV/cm where the denominator represents the spacing between electrodes 25 in the reservoir 21 in cm. The nsEPs may have a bipolar voltage ranging from +/−12.5 kV/cm to +/−25 kV/cm and a monopolar voltage ranging from +12.5 kV/cm to +25 kV/cm.

As the energy imparted from each nsPEF decreases, the number of pulses in the pulse count may increase to compensate. The total pulse count per treatment may range from 1 pulse to 1000 pulses, and preferably from 10 pulses to 100 pulses.

The nsPEF generator 23 may be connected to each of the two electrodes 25 by a dedicated electrical lead 26, such as a wire. Alternatively, delivery of the nsPEF to the electrodes 25 may be achieved b electrical leads 26 comprising a spark gap switch that discharges over an air gap between two conductive plates completing the circuit.

The electrodes 25 may be aluminum cuvette plates having a contact resistance between the aluminum cuvette electrode 25 plates/solution 22 of 8 ohms. The electrodes 25 may have a gap therebetween of 1 mm to 4 mm and preferably 2 mm. The reservoir 21 may be a cuvette having a volume of 200 microliters to 800 microliters and particularly 500 microliters (mL). The cuvette may have opposed metallic sidewalls, particularly aluminum sidewalls which function as the electrodes 25.

The solution 22 may be a bovine coronavirus (BCoV), usable as a surrogate for the severe acute respiratory syndrome coronavirus 2 (SARS-COV-2) virus. Bovine coronavirus (BCOV) NR-445, Mebus available from BEI Resources of Manassas, VA has been found suitable for test purposes, although one of skill will recognize that the coronavirus in practice will be whatever occurs on site. This batch system apparatus 20 provides the benefit that the nsPEF can be tailored to a specific solution 22 and coronavirus.

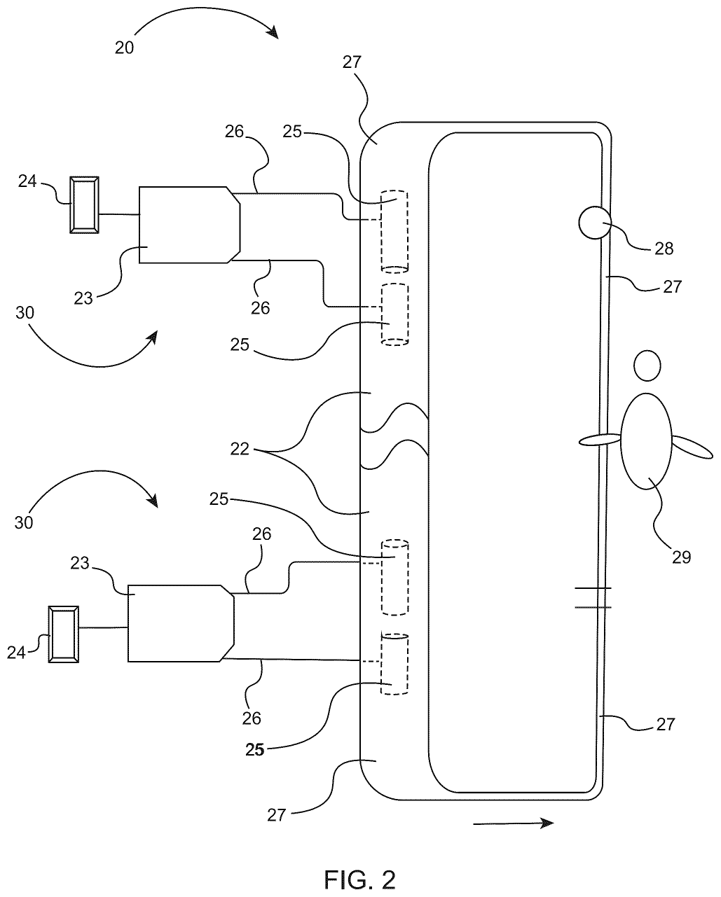

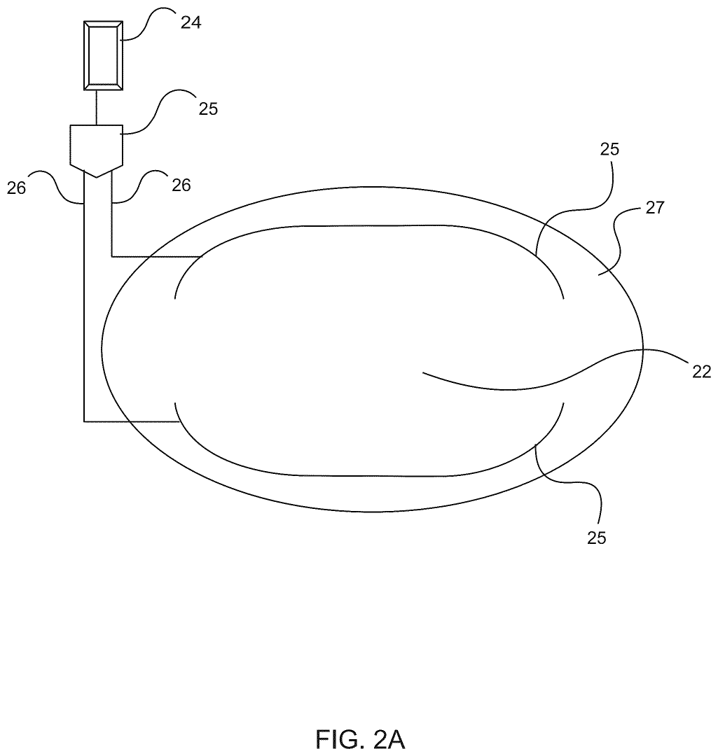

Referring to FIG. 2, the invention may comprise an apparatus 20 which provides a dynamic system. The dynamic system provides for open loop flow, and preferably closed loop flow, in a pipe 27. A pump 28 may be used in known fashion to provide a pressure differential to effect the flow of the solution 22 as described herein. The pump 28 may be energized by the same power supply 24 used for the nsPEF generator 23 or by a separate power supply (not shown). The pump 28 may particularly propel the solution 22 from an outlet of the reservoir 21, through one or more stages 30 as described below, and through an inlet back into the reservoir 21 thereby forming a closed loop. The closed loop supplies solution 22 from the reservoir 22

The solution 22 in this apparatus 20 passes through at least one stage 30 in the flow path. As shown, each stage 30 may comprise a dedicated power supply 24 and nsPEF generator 23. The nsPEF generator 23 is connected to paired electrodes 25 by the aforementioned electrical leads 26. Alternatively, a single power supply 24 may provide energy for plural stages 30. Likewise, a single nsPEF generator 23 may charge plural pairs of electrodes 25.

If plural stages 30 are used, the stages 30 may be mutually identical. This arrangement provides the benefit of simplicity of construction and inventory. Alternatively, plural stages 30 may be mutually different. This arrangement provides the benefit that different stages 30 can be tailored to specific needs as judged helpful for future use. For example, the stages 30 may be disposed in series, as shown, with successive stages 30 selectively energized for imparting more nsPEF, as may be needed to deactivate more virulent virus strains in the solution 22.

The electrodes 25 may be disposed within the pipe 27, be disposed outside of the pipe 27 and particularly circumscribe the pipe 27 or disposed in any other configuration which provides for an effective amount of nsPEF energy from the nsPEF generator 23 to reach the solution 22 and deactivate the virus in the solution 22. The electrodes 25 may be disposed in series, may be coaxial, etc.

If desired, the reservoir 21 may have an inlet/outlet configuration, obviating batch treatment in favor of continuous treatment. This dynamic system apparatus 20 provides the benefit that solution 22 can be treated more efficiently and with less handling than when using a batch system.

Prophetically, the reservoir 21 may be an animal, more particularly a mammal and even more particularly a human 29. In this embodiment, the dynamic apparatus 20 may provide for sanitizing blood from coronavirus. The solution 22 is the blood and the pipe 27 may be in the form of an intravenous circulation, similar to what is used for blood transfusions. The blood leaves the human 29 either under systolic pressure or with assistance from the pump 28. The blood passes through one or more stages 30 as needed for adequate neutralization of the coronavirus then returns to the human 29 circulatory system in the closed loop. This arrangement provides the benefits that the stages 30 may be remote from the human 29, and that one or more stages 30 may be used as needed providing for a modular construction.

One of skill will understand that as the flow rate, either mass flow rate or flow velocity increases, more nsPEF energy will need to be imparted using the aforementioned parameters. Likewise, the shape of the pipe 27 may influence the nsPEF energy required for efficacy.

Referring to FIG. 2A, if desired, the pipe 27 may have a non-round cross section to decrease the hydraulic radius. This arrangement provides for increased surface area of the paired, spaced apart electrodes 25 per unit length of the pipe 27. Such electrodes 25 may be spaced apart in the radial direction as shown whether diametrically opposed as shown or diametrically offset.

The increased surface area of the electrodes 25 provides for greater contact and improved proximity of the solution 22 with the electrodes 25, prophetically increasing the efficacy of the nsPEF. An advantage of any of the aforementioned apparatus 20 and methods described and claimed herein is that boiling can unexpectedly and advantageously be avoided. Boiling is undesirable due to the safety hazards and the extra energy needed to overcome the latent heat of vaporization leading to inefficiency.

To verify the efficacy of the apparatus 20 and methods described and claimed herein, the following experiments and modeling were conducted. In all experiments Homo sapiens ileocecal colorectal adenocarcinoma cells (HRT-18G, ATCC No. CRL-11663), were obtained from American Type Culture Collection (ATCC) (Manassas, Virginia). HRT-18G cells were maintained in Dulbecco's Modified Eagle's Medium (DMEM) supplemented with 5% fetal bovine serum (FBS) and 100 U/ml penicillin/streptomycin at 37° C. with 5% CO2 in air. The media and its components were purchased from ATCC (Manassas, Virginia). Bovine coronavirus (BCoV) NR-445, Mebus was acquired from BEI Resources (Manassas, Virginia). All experiments containing BCOV were conducted in Eagle's Minimum Essential Medium (EMEM, ATCC) supplemented with 100 U/ml penicillin/streptomycin. BCoV was propagated to create a virus stock via passage in host (HRT-18G) cells. HRT-18G cells were grown to confluent monolayers in 6-well plates. Prior to infection, cells were rinsed twice with serum free media (SFM) and inoculated with 200 μL of BCOV BEI stock. Virus-inoculated cells were incubated for 2 h at 37° C., 5% CO2, 95% RH, then 1.8 ml of EMEM containing 2% FBS was added to each well. The cells were then incubated for 6 days (37° C., 5% CO2, 95% RH) to allow infection, as evidenced by the appearance of cytopathic effects (CPE). This process was repeated twice with reinfection in successively larger cell culture vessels to create a large volume of BCOV stock for this study. BCOV stock was aliquoted in small volumes and frozen at −80° C. until use.

BCoV was exposed to MP (600 ns) or BP (300+300 ns) pulses. Then 450 μL of BCOV in solution 22 was added to a conventional electroporation cuvette with a 2-mm gap. 600 nsPEF exposures were performed for 0 to 1000 pulses at an amplitude of 12.5 or 25 kV/cm and a 1 Hz repetition rate. Two Marx bank capacitor systems were used to generate either the 600 ns MP or 300+300 ns BP pulse. A high voltage power supply 24 was used to charge the Marx bank. Delivery to the cuvette was achieved by a spark gap switch that discharged over an air gap between two conductive plates completing the circuit. The rate of discharge and amplitude was set by adjusting the charging voltage and the distance between the plates, respectively. The pulse delivered to the cuvette was measured using a high voltage probe connected to a high-speed oscilloscope (TDS3052B, Tektronix®, Beaverton, OR). Removal of the charging voltage controlled the number of pulses delivered, which were manually counted.

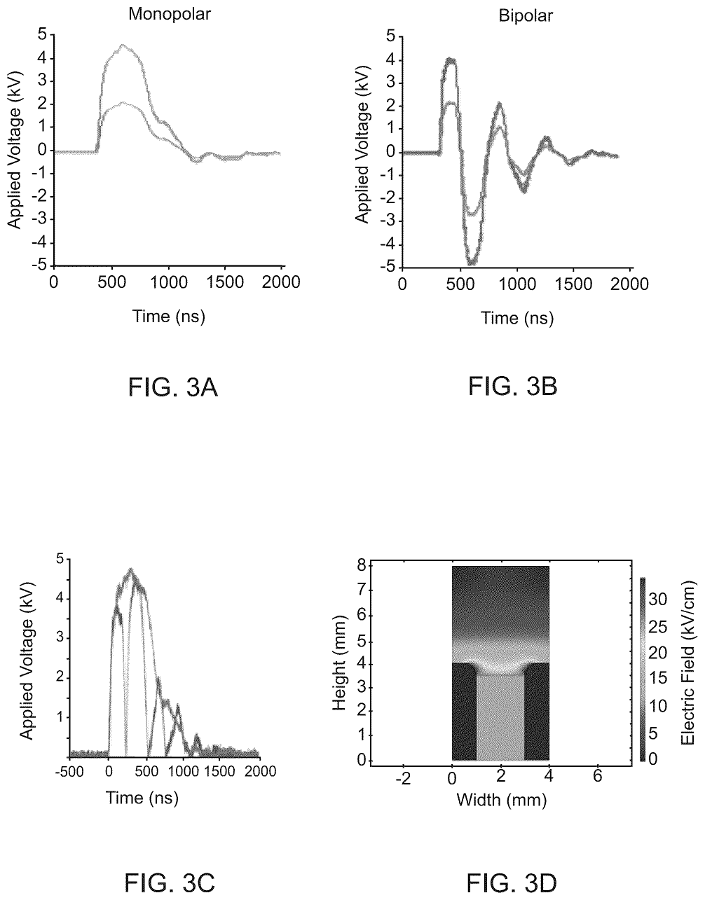

Referring to FIGS. 3A-3D, the resultant pulse shapes are shown in FIG. 3A (MP) and FIG. 3B (BP). Superimposition of the 25 kv/cm MP and BP pulses is shown in FIG. 3C. For all exposures, the peak of the highest amplitude BP component was matched to the peak amplitude of the MP pulse.

For both exposure systems, COMSOL Multiphysics® software v. 5.4 (COMSOL) was used to model electrodynamics and temperature within the exposure cuvette. The predicted E-field spatial distribution displays uniformity throughout the exposure solution 22 (FIG. 3D) and, therefore, the E-field was assumed to be spatially uniform throughout the BCoV exposures.

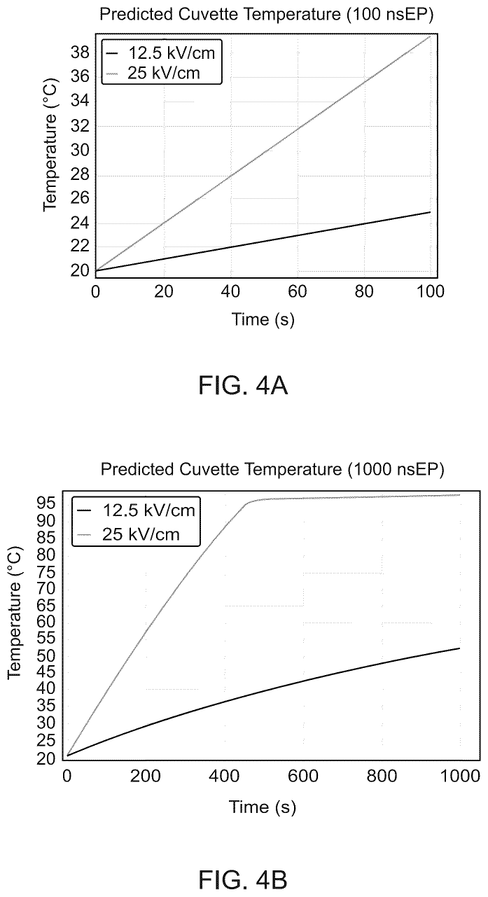

Referring to FIG. 4A and FIG. 4B, temperature rise during the exposure was predicted using COMSOL Multiphysics modeling software. Phase change in the media (i.e., boiling) is expected after approximately 500 pulses. A solution 22 conductivity of approximately 1.3 S/m and a contact resistance between the aluminum cuvette electrode 25 plates/solution 22 of 8 ohms was assumed. The solution 22 reaches near boiling temperatures of approximately 100° C., limiting the continued rise in temperature. Pulse to pulse cooling increases as temperature rises because of the heat capacity of the solution 22. To validate the model predictions an Opsens probe (OpSens, Inc., Quebec, Canada) was used to measure temperature within the cuvette. Prior to exposure, initial temperature of the BCOV solution 22 in the electroporation cuvette was measured and recorded. After the specified exposure duration, the cuvette was quickly removed from the system, the cap removed, and an Opsens probe was inserted to evaluate the temperature rise following exposure. It can be seen that for both 100 and 1000 MP pulses at either 12.5 kV/cm and 25 kV/cm, boiling is advantageously predicted to be avoided.

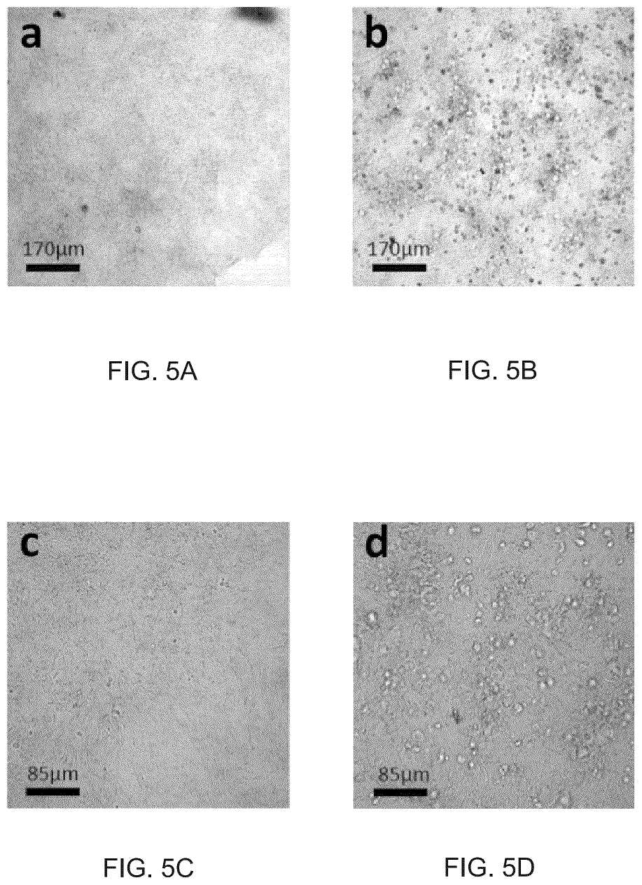

Referring to FIGS. 5A-5D, after nsPEF exposure each BCOV sample was diluted from 10⁰ to 10⁻⁴ in SFM and added to HRT-18G cells (plated at 15,000 cells per well in 96 well plates 24 h before) for virus titer determination to obtain the 50% tissue culture infectious doses (TCID₅₀). Prior to infection, cell monolayers were rinsed twice with SFM, and then inoculated with 20 μL of diluted BCOV samples. Virus-inoculated cells were incubated for 1 h at 37° C., 5% CO₂, 95% RH, then 80 μL of EMEM containing 3% FBS was added to each well. The cells were then incubated for 6 days (37° C., 5% CO2, 95% RH) to allow infection to occur. On Day 6 post-infection, three independent researchers evaluated each well microscopically to determine if CPE was present.

FIGS. 5B and 5D both show the appearance of cells infected with virus at day 6 (Virus) in which CPE is evident vs controls (No Virus) in FIG. 5A and FIG. 5C that were incubated with media (control) showing a normal appearance. Researchers were blinded to the identity of the samples and results were evaluated as binary (i.e., infected or not infected). After evaluation, the titer of infectious virus was quantified by calculating the TCID50/ml for each condition. TCID50/ml was calculated using the Reed-Muench equation. To compare results between multiple experiments (where definite TCID50/ml values may differ), the TCID50/ml values were converted to percentage Inactivation (ratio), as according to:

$$\% Infectivity = \{ TCID 50 / ml Sample ( CPE ) / TCID 50 / ml Sham ( CPE ) \} \times 100$$

$$and$$

$$\% Inactivation ( Radio ) = 100 - \% Infectivity .$$

The data show 100 MP pulses yields 70.3+/−4.677% inactivation and 100 BP pulses yields 74.0+/−5.647% inactivation.

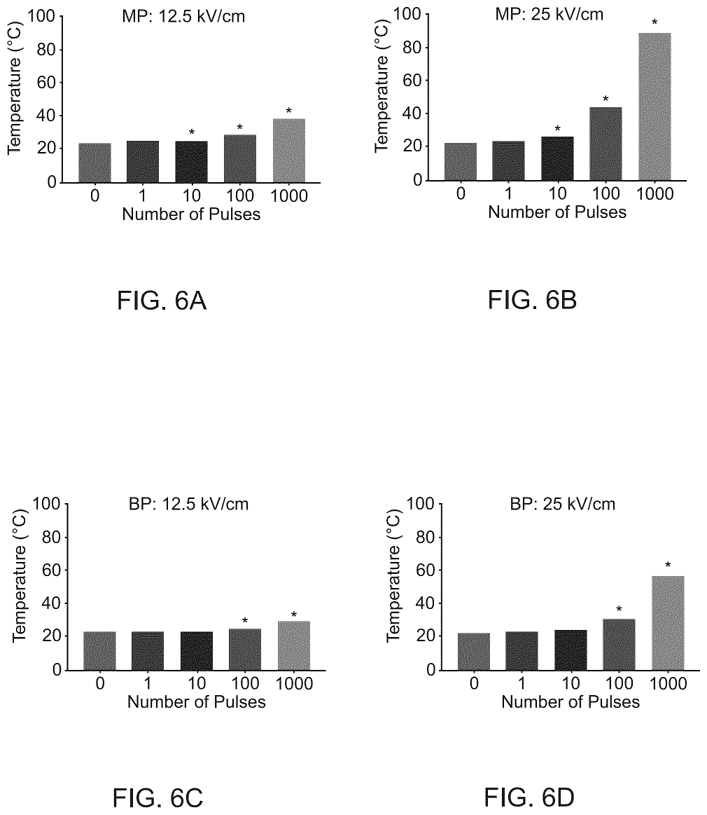

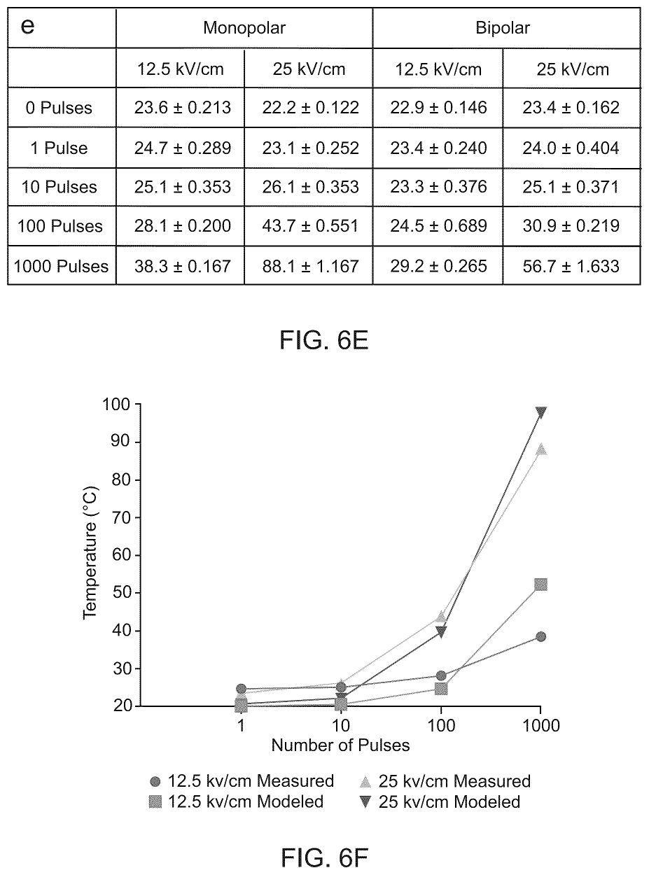

Referring to FIGS. 6A-6D, to better evaluate the potential hypotheses that could explain the noted differences between MP and BP exposure, the mechanical forces acting on the viral particle in solution 22 induced by a similar, analytically defined, electric field were estimated. The model electric field pulse was decomposed into frequency components and independently solved for each frequency component of the model electric pulse. The net charging solution 22 was than reconstructed through simple superposition of each component.

The virion equations of motion were numerically solved with Mathematica (Mathematica, Princeton, New Jersey) using Stokes' law, i.e., assuming the virion displacement was small, and the motion was sufficiently smooth. The virion is assumed to have a net charge on the order of 5×10⁻¹⁵ Coulombs for a naive estimate based on the length of negatively charged RNA contained in a virion, 1 elementary charge per base-pair for 30 kilobase-pairs and mass of 1 fg. In one-dimension this relationship is given by:

$$dx ( t ) / dt = v ( t ) ,$$

$$[ dv ( t ) / dt ] = q E ( t ) / m - [ \partial / m ] * v ( t ) ,$$

where x(t) is the virion position over time, v(t) is the virion velocity, q is the virion charge, ∂ is the drag coefficient of a 50 nm sphere and m is the virion mass.

All experiments were performed in triplicate and a mean and standard error of the mean (S.E.M.) were calculated for each experiment. Error bars are provided as +/−S.E.M. Pairwise comparisons of virus titers (TCID50/ml) or % infectivity for each nsPEF exposure were compared to control/sham (0 pulses) using pairwise Student's t-test. The criterion for significance was set at p<0.05 for a type I error. The temperature of the BCoV solution 22 (EMEM) was measured for each amplitude and pulse train used in this study.

The results showed that increasing the pulse number increased the overall temperature within the sample, for each respective exposure. Additionally, increasing the amplitude of the pulses enhanced the thermal gradient during exposure (FIG. 6A vs FIG. 6C and FIG. 6B vs FIG. 6D). MP pulses produced greater overall temperature rises compared to their matched BP pulsed conditions (FIG. 6A vs FIG. 6C and FIG. 6B vs FIG. 6D).

The rise in postexposure measured temperature also corelates with that predicted by the COMSOL modeling, as shown for MP pulses performed for the 12.5 kV/cm and 25 kV/cm amplitudes (FIG. 6F), further corroborating the results. Therefore it is believed the total energy in each exposure is different due to the phase changes in the bipolar exposures resulting in a lower achieved temperature for bipolar exposures of the same pulse number and amplitude. These experiments indicate the condition that generated heat capable of completely inactivating BCOV was 1000 MP pulses at an amplitude of 25 kV/cm.

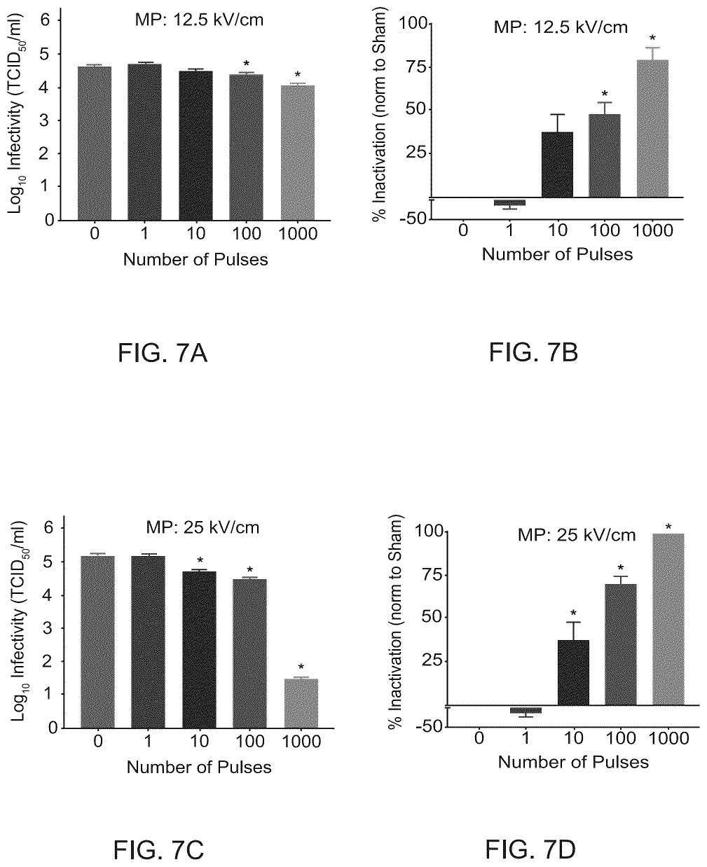

Referring to FIG. 7A-7D, the inactivation of BCOV was evaluated by assessing viral titer (TCID50/ml) following exposure to 600 ns MP pulses (0, 1, 10, 100, or 1000) at two amplitudes (12.5 kV/cm or 25 kV/cm) based on the formation of CPE in cultured cells. Viral titers were determined upon titration on HRT-18G cells and evaluated at 6 days post inoculation. a and b are the TCID50/ml and % Inactivation (i.e., data normalized to non-exposed virus sham control/0 pulses) following exposure to 12.5 kV/cm MP pulses. FIG. 7C and FIG. 7D represent the TCID50/ml and % Inactivation following exposure to 25 kV/cm MP pulses. Data are expressed as mean values+S.E.M. of at least three independent experiments (n=3). Statistically significant differences are noted by an asterisk, which represents p<0.05. For the 12.5 kV/cm, the results show statistically significant BCOV inactivation starting at 100 pulses (FIG. 7A and FIG. 7B). In the higher amplitude field, inactivation of BCOV was significant following exposure to 10 pulses (FIG. 7C and FIG. 7D). In both conditions, the inactivation increased as amplitude and/or pulse number increased, with maximal “% inactivation” at 1000 pulses for both amplitudes.

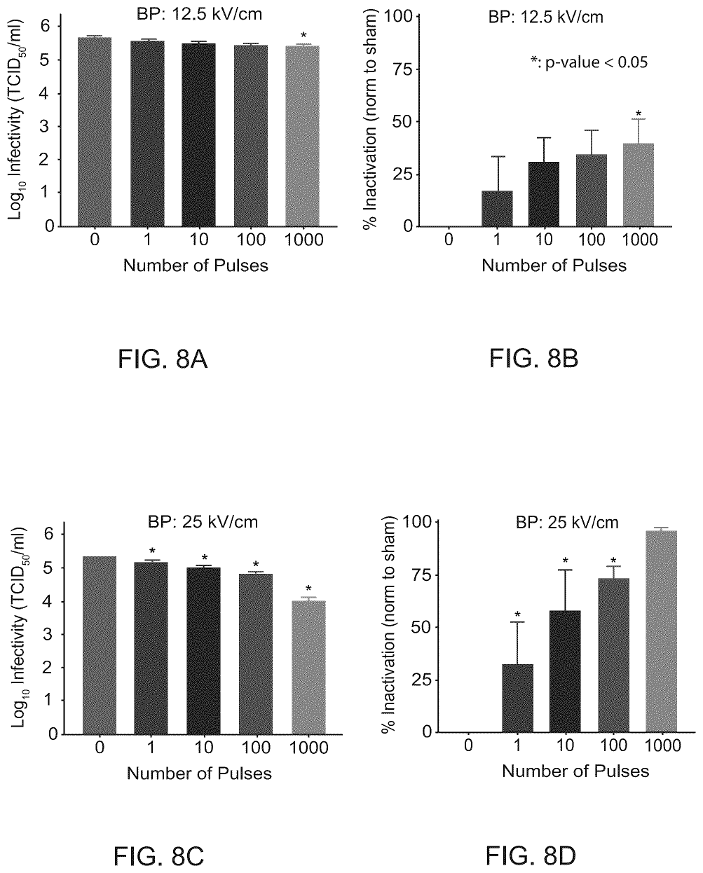

Referring to FIGS. 8A-8D, BP pulses, which are characterized by a reversal of polarity halfway through the pulse duration, have been shown to be as effective as MP exposures for micro and millisecond pulse durations. However, for nanosecond duration pulses, BP pulses are markedly less effective as compared to energy-matched MP pulses. Using second harmonic imaging, it was shown that BP nsPEF exposures impact both sides of a mammalian cell similarly to longer pulses but fail to induce as significant permeabilization. Viral infectivity was assessed by CPE evaluation. Viral titers were determined upon titration on HRT-18G cells, as described in the methods, and evaluated at 6 days post inoculation. FIG. 8A and FIG. 8B represent the TCID50/ml and % Inactivation (i.e., data normalized to non-exposed virus sham control/0 pulses) following exposure to 12.5 kV/cm BP pulses. C and d are the TCID50/ml and % Inactivation following exposure to 25 kV/cm BP pulses. Data are expressed as mean values+S.E.M. of at least three independent experiments (n=3). Statistically significant differences are noted by an asterisk, which represents p<0.05. The mechanism behind “BP cancellation” remains unknown. As BP exposures match free-field exposures, BCoV was exposed to BP (300+300 ns) pulses as a comparison to 600 ns MP pulses. As shown in FIGS. 3A-3C and FIGS. 6A-6D, the overall energy within the BP pulse is ˜ 30% less than the MP pulse. Peak electric field amplitudes were matched, hypothesizing that peak electric field amplitude would be the driver of virus inactivation. Notably, the temperature profile for BP pulse exposure shows less temperature increase at both amplitudes tested (FIG. 6C-FIG. 6D). BCoV to was exposed to BP pulses (0, 1, 10, 100, or 1000) at two amplitudes (12.5 kV/cm or 25 kV/cm) and evaluated the infectivity of exposed virion based on the formation of CPE in cultured cells. Results shows statistically significant inactivation of BCOV at the 1000 pulse condition at low (12.5 kV/cm) amplitude (FIG. 8A and FIG. 8B). Statistically significant inactivation of BCOV at 1 pulse of 25 kV/cm nsPEF occurs with increasing pulse numbers (FIG. 8C and FIG. 8D).

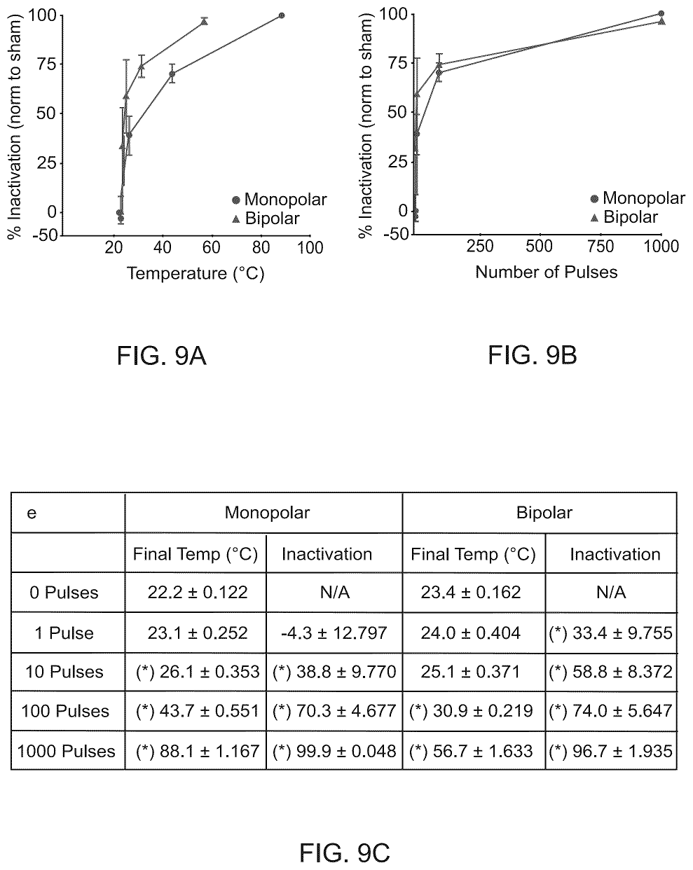

Referring to FIGS. 9A-9C, to differentiate between the properties of nsPEF that induces inactivation, we plotted BCOV infectivity data (MP or BP pulses) at 25 kV/cm as a function of sample temperature (FIG. 9A) or pulse number (FIG. 9B). Data are expressed as mean values±S.E.M. of at least three independent experiments (n=3). In FIG. 9C, statistically significant differences are noted by an asterisk, which represents p<0.05. The results unexpectedly and advantageously show that BP pulsing induces inactivation of BCoV at lower temperatures than MP pulsing equivalents.

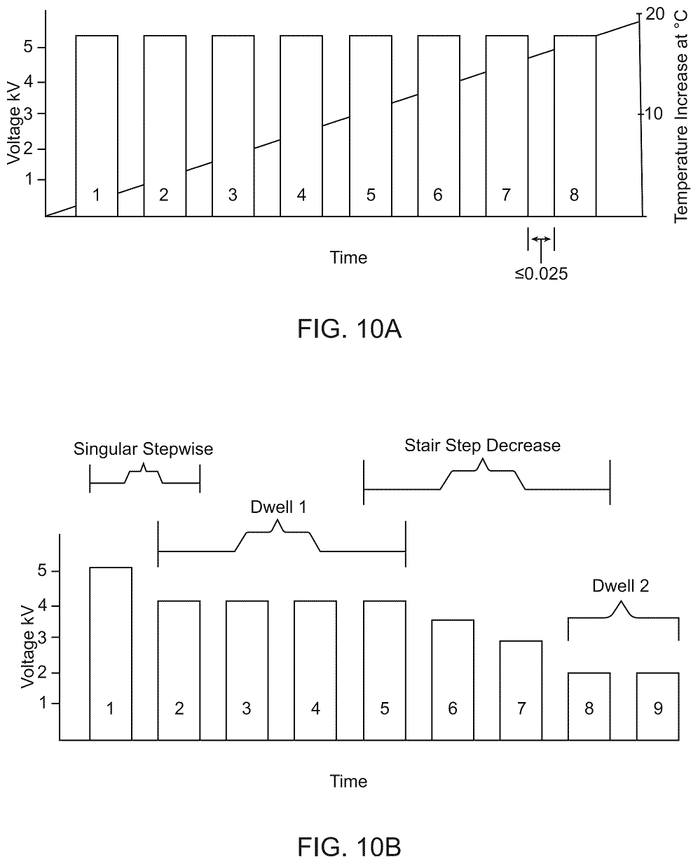

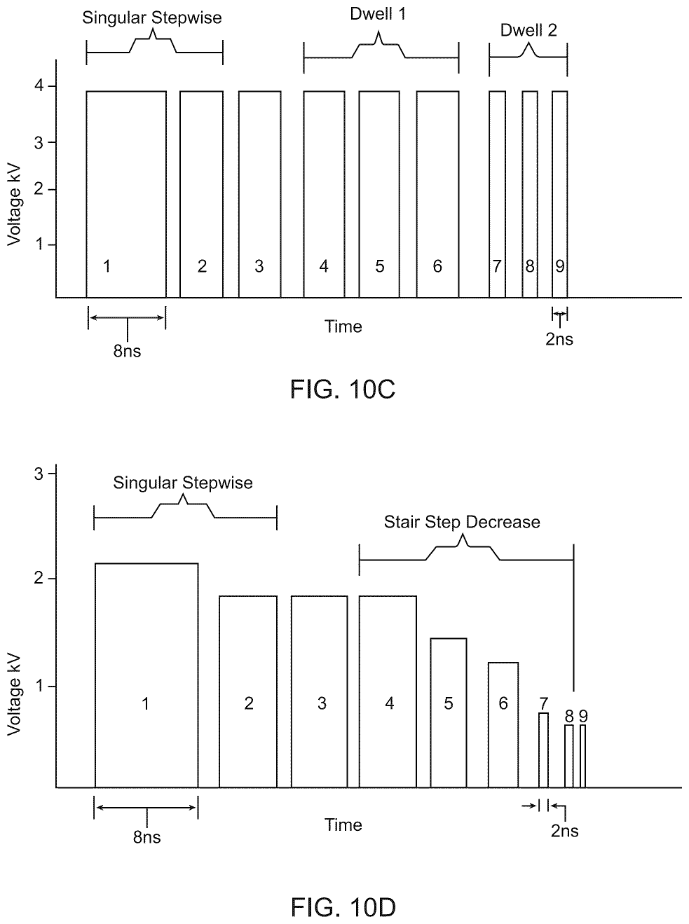

Referring to FIGS. 10A-10D, the method according to the present invention may impart from 1500 kVns to 3500 KVns and preferably from 1800 kVns to 3200 kVns to effectively deactivate or sterilize 200 mL to 800 mL of coronavirus according to the present invention. The process may deploy from 2 pulses to 8 pulses and optionally from 3 pulses to 5 pulses. The nsEPs may have a duration of 2 ns to 8 ns and optionally 3 ns to 5 ns. The nsEPs may have a voltage ranging from 2 kV to 5 KV and optionally and optionally 3 kV to 4 kV.

During this process boiling is advantageously avoided. The temperature increase of the coronavirus solution in the reservoir may be less than 20 degrees C. and preferably less than 10 degrees C. The temperature rise may be linear or nonlinear.

As used herein monotones, both increasing and decreasing, include dwell, stepwise changes, slope changes, second, third and fourth order changes, etc. I.e., monotonically increasing includes both dwell and increases during the same process. Monotonically decreasing includes both dwell and decreases during the same process. As used herein dwell and my follow a singular stepwise decrease, while a stairstep decrease/increase indicates each nsEP changes in energy from the previous nsEP.

Referring particularly to FIG. 10A, according to one method of the present invention, the nsEPs may have constant energy input with each pulse. I.e. the pulses may be of constant voltage and constant duration. The method provides the benefit of simplicity and more predictable temperature increase.

Referring particularly to FIG. 10B, according to one method of the present invention, the first nsEP (labelled 1) may provide more energy than subsequent nsEPs. The increased energy may be accomplished with greater voltage at constant pulse duration. Subsequent pulses (2, 3, 4, 5) may have a singular stepwise voltage decrease from the first pulse. And other pulses (5, 6, 7, 8) may have a stairstep voltage decrease from the previous nsEP. This method is believed to be advantageous due to the large first insult beginning the deactivation.

Referring particularly to FIG. 10C, according to yet another method of the present invention, the first nsEP (labelled 1) may provide more energy than subsequent nsEPs. The increased energy may be accomplished with greater pulse duration at constant voltage. Subsequent pulses (2, 3, 4, 5, 6) may have a singular stepwise pulse duration decrease from the first pulse. And other pulses (7, 8, 9) may have another singular stepwise pulse duration decrease from the previous pulse duration. This method is believed to be advantageous due to the large first insult beginning the deactivation.

Referring particularly to FIG. 10D, according to yet another method of the present invention, the first nsEP (labelled 1) may provide more energy than subsequent nsEPs in a hybrid fashion. After the first nsEP, subsequent nsEPs (2, 3, 4) may have both a lesser voltage and lesser pulse duration than the first nsEP in a single stepwise decrease. Subsequent pulses (5, 6, 7) may have a stairstep decrease in voltage and/or pulse duration. Still further subsequent nsEPs (8,9) may have yet another singular stepwise decrease.

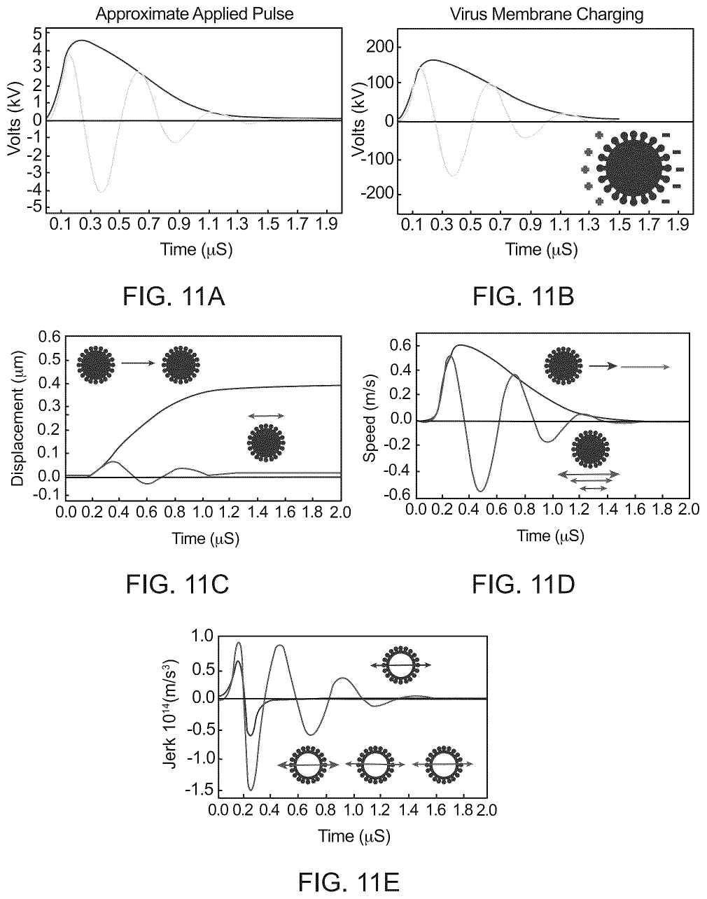

Referring to FIGS. 11A-11E, results of the charging model are illustrated. The applied voltage and the resultant charging on the viral membrane are presented in FIG. 11A and FIG. 11B. The inset depicts the viral membrane depicts the charging direction of the viral membrane (which oscillates for bipolar exposures). The results of the mechanical models are presented in FIG. 11C, FIG. 11D and FIG. 11E. The insets illustrate the direction of the physical forces on the viral membrane.

All values disclosed herein are not strictly limited to the exact numerical values recited. Unless otherwise specified, each such dimension is intended to mean both the recited value and a functionally equivalent range surrounding that value. For example, a dimension disclosed as “40 mm” is intended to mean “about 40 mm.” The term “or” as used herein is to be interpreted as an inclusive or meaning any one or any combination. Therefore, “A, B or C” means “any of the following: A; B; C; A and B; A and C; B and C; A, B and C.” Every document cited herein, including any cross referenced or related patent or application, is hereby incorporated herein by reference in its entirety unless expressly excluded or otherwise limited. The citation of any document or commercially available component is not an admission that such document or component is prior art with respect to any invention disclosed or claimed herein or that alone, or in any combination with any other document or component, teaches, suggests or discloses any such invention. Further, to the extent that any meaning or definition of a term in this document conflicts with any meaning or definition of the same term in a document incorporated by reference, the meaning or definition assigned to that term in this document shall govern according to Phillips v. AWH Corp., 415 F.3d 1303 (Fed. Cir. 2005). All limits shown herein as defining a range may be used with any other limit defining a range of that same parameter. That is the upper limit of one range may be used with the lower limit of another range for the same parameter, and vice versa. As used herein, when two components are joined or connected the components may be interchangeably contiguously joined together or connected with an intervening element therebetween. A component joined to the distal end of another component may be juxtaposed with or joined at the distal end thereof. While particular embodiments of the present invention have been illustrated and described, it would be obvious to those skilled in the art that various other changes and modifications can be made without departing from the spirit and scope of the invention and that various embodiments described herein may be used in any combination or combinations. It is therefore intended the appended claims cover all such changes and modifications that are within the scope of this invention.

What is claimed is:

1. A method of viral sterilization comprising the steps of:
providing a coronavirus in a reservoir;
providing two spaced apart electrodes in electrical communication with the coronavirus and being connected to an electrical power supply; and
applying an effective amount of nanosecond duration electric pulses (nsEPs) from the power supply to thereby substantially deactivate the coronavirus.

2. A method according to claim 1 wherein the step of applying an effective amount of nsEPs comprises applying bipolar nsEPs.

3. A method according to claim 2 wherein the step of applying an effective amount of nsEPs comprises applying bipolar nsEPs having a bipolar voltage ranging from +/−12.5 kV/cm to +/−25 kV/cm.

4. A method according to claim 1 wherein the step of applying an effective amount of nsEPs comprises applying 1 pulse to 1000 pulses.

5. A method according to claim 3 wherein the step of applying an effective amount of nsEPs comprises applying 10 pulses to 100 pulses.

6. A method according to claim 4 wherein the step of applying an effective amount of nsEPs comprises applying nsEPs for a period ranging from 250 ns+250 ns to 350 ns+350 ns.

7. A method according to claim 6 wherein the step of applying an effective amount of nsEPs comprises applying nsEPs at a frequency of 0.9 Hz to 1.1 Hz.

8. A method according to claim 4 further comprising the steps of:
depositing a solution of bovine coronavirus within the reservoir; and
not boiling the solution.

9. A method of selective coronavirus sterilization comprising the steps of:
providing 400 microliters to 500 microliters of bovine coronavirus in a reservoir;
providing two spaced apart electrodes in electrical communication with the coronavirus and being connected to an electrical power supply; and
applying an effective amount of nanosecond duration monopolar electric pulses (nsEPs) from the power supply to thereby substantially deactivate the coronavirus.

10. A method according to claim 9 wherein the step of applying an effective amount of nsEPs comprises applying nsEPs at a frequency of 0.9 Hz to 1.1 Hz.

11. A method according to claim 10 wherein the step of applying an effective amount of nsEPs comprises applying nsEPs for a period ranging from 500 ns to 700 ns.

12. A method according to claim 11 wherein the step of applying an effective amount of nsEPs comprises applying 1 pulse to 1000 pulses.

13. A method according to claim 12 wherein the step of applying an effective amount of nsEPs comprises applying nsEPs having a voltage ranging from +12.5 kV/cm to +25 kV/cm.

14. A method according to claim 12 further comprising the steps of:
depositing a solution of bovine coronavirus within the reservoir; and
boiling the solution with less than 1000 nsEPs.

15. An apparatus for viral sterilization of coronavirus comprising:
a reservoir for containing a coronavirus solution therein; and
a power supply in electrical communication with two spaced apart electrodes configured to apply an electric current to a coronavirus solution when contained within the reservoir, the power supply being able to provide upon demand a nanosecond duration electric pulses (nsEPs) effective to sterilize an object disposed in the reservoir from a coronavirus.

16. An apparatus according to claim 15 wherein the power supply comprises at least one Marx bank capacitor.

17. An apparatus according to claim 16 wherein the power supply provides a bipolar voltage ranging from +/−12.5 kV/cm to +/−25 kV/cm.

18. An apparatus according to claim 17 wherein the power supply provides a bipolar voltage for a period ranging from 250 ns+250 ns to 350 ns+350 ns.

19. An apparatus according to claim 16 wherein the electrodes are spaced apart a distance ranging from 1 mm to 4 mm.

20. An apparatus according to claim 19 wherein the reservoir comprises a plurality of aluminum cuvettes electrically joined to the power supply in parallel.
